(19) United States
(12) Patent Application Publication
(10) Pub. No.: US 2009/0233892 A1
(43) Pub. Date: Sep. 17, 2009

(54) Topical Compositions Comprising Hydrocortisone Butyrate

(75) Inventors: Nilendu Sen, Navi Mumbai (IN); Kusum Gole, Satara (IN); Manoj Soni, Navi Mumbai (IN)

Correspondence Address: GLENMARK PHARMACEUTICALS INC USA, 750 CORPORATE DRIVE, MAHWAH, NJ, 07430, US

(73) Assignee: GLENMARK GENERICS LTD, Mumbai (IN)

(21) Appl. No.: 12/400,935

(22) Filed: Mar. 10, 2009

Related U.S. Application Data
(60) Provisional application No. 61/134,274, filed on Jul. 8, 2008.

(30) Foreign Application Priority Data
Mar. 11, 2008 (IN) 495/MUM/2008

(51) Int. Cl. A61K 31/57

(52) U.S. Cl. 514/179

(57) ABSTRACT

The present invention generally relates to a a topical composition, such as a dermatological cream, containing the corticosteroid compound hydrocortisone butyrate.

### PRIORITY

This application claims priority to Indian Provisional Application No. 495/MUM/2008, filed on Mar. 11, 2008, and under U.S.C. §119 (e) to U.S. Provisional Application No. 61/134274, filed on Jul. 8, 2008; the contents of which, are hereby incorporated by reference.

### BACKGROUND OF THE INVENTION 1. Technical Field

The present invention generally relates to a topical pharmaceutical composition containing the corticosteroid compound hydrocortisone butyrate. 2. Description of the Related Art

Suitable hydrocortisone diesters are known and are specified in, for example, DE2,910,899 and DE2,826,257. Examples of such esters are hydrocortisone 17-propionate 21-acetate, hydrocortisone 17-butyrate 21-acetate, hydrocortisone 17, 21-dipropionate, hydrocortisone 17-propionate 21-butyrate, and hydrocortisone 17-butyrate 21-propionate.

Hydrocortisone butyrate, also known as 17α-butyryloxy-21 -propionyloxy-11β-hydroxy4-pregnene-3,20-dione, is a known corticosteroidal compound having excellent anti-inflammatory activity and is topically administrable with little side-effects (see, for example, U.S. Pat. No. 4,290,962). Hydrocortisone butyrate is sold under the LOCOID® tradename as a cream, ointment or solution.

U.S. Pat. No. 5,635,497 discloses a topical application composition in the form of a fatty cream comprising 50 to 80% by weight of fatty components, 1.5 to 5% by weight of at least one hydrophilic non-ionic surfactant, about 6% of fatty alcohols and esters and a therapeutically effective amount of at least one topically active therapeutic agent and water.

### SUMMARY OF THE INVENTION

The present invention provides a stable oil-in-water fatty cream composition comprising hydrocortisone butyrate and one or more fatty components.

The present invention provides a stable oil-in-water fatty cream composition comprising hydrocortisone butyrate and one or more fatty components, wherein said composition contains a greater amount of fatty components than water.

The present invention provides a topical cream comprising (a) about 0.005 wt. % to about 1.0 wt. % hydrocortisone butyrate; and (b) at least 6 wt. % of a fatty alcohol, ester thereof or a mixture thereof.

The present invention provides a topical cream comprising (a) about 0.005 wt. % to about 1.0 wt. % hydrocortisone butyrate; (b) about 6.0 wt. % to about 24.0 wt. % a fatty alcohol, ester thereof or a mixture thereof (c) about 50 wt. % to about 80 wt. % fatty component; (d) about 1.0 wt. % to about 5.0 wt. % of at least one surfactant; and the balance being water, preferably purified water, USP.

The present invention provides a stable oil-in-water fatty cream composition comprising hydrocortisone butyrate and two or more surfactants.

The present invention also provides a topical cream comprising hydrocortisone butyrate and two or more surfactants which provide a combined HLB to the composition, of about less than about 14.

The present invention also provides a topical cream comprising (a) about 0.005 wt. % to about 1.0 wt. % hydrocortisone butyrate; and (b) two or more surfactants which provides combined HLB to the composition, of about less than about 14.

The present invention also provides a topical cream comprising (a) about 0.005 wt. % to about 1.0 wt. % hydrocortisone butyrate; and (b) two or more surfactants which provides combined HLB to the composition, of about less than about 14, wherein fatty alcohol, ester or a mixture thereof is less than 6.0 wt. %.

The present invention provides a topical cream comprising (a) about 0.005 wt. % to about 1.0 wt. % hydrocortisone butyrate; (b) about 2.0 wt. % to about 4.0 wt. % a fatty alcohol, ester thereof or a mixture thereof; (c) about 50 wt. % to about 80 wt. % fatty component; (d) about 1.0 wt. % to about 7.0 wt. % of two or more surfactants having HLB of blend less than about 14; and the balance being water, preferably purified water, USP

The present invention provides a topical cream comprising (a) about 0.1 wt. % hydrocortisone butyrate; (b) about 1.0 wt. % to about 7.0 wt. % of two or more surfactants having HLB of blend between 8.5 to 14, preferably between 9 to 11 and (c) about 2.0 wt. % to about 4.0 wt. % of a fatty alcohol, ester thereof or a mixture thereof.

### DETAILED DESCRIPTION OF THE INVENTION

As described previously, the present invention relates to topical composition such as a dermatological cream containing the corticosteroid compound hydrocortisone butyrate. The topical cream of the present invention contains about 0.005 wt. % to about 1.0 wt. % hydrocortisone butyrate.

The cream composition of the present invention is preferably a stable oil-in-water fatty cream that comprises hydrocortisone butyrate and one or more fatty components. The present invention contains a greater amount of fatty components than water.

One embodiment of the present invention is directed to a stable topical cream containing at least hydrocortisone butyrate in an amount of from about 0.005 wt. % to about 1.0 wt. %; a fatty alcohol, ester thereof or a mixture thereof in amount of from about 6.0 wt. % to about 24.0 wt. %, fatty components in an amount of from about 50 wt. % to about 80 wt. %; at least one surfactant in an amount of about 10 wt. % to about 5.0 wt. %; and the balance in water, preferably, USP purified water and wherein the cream contains a greater amount of fatty components than water. Preferrably, the present invention provides a topical cream comprising (a) about 0.1 wt. % hydrocortisone butyrate; and (b) about 10.0 wt. % to about 20.0 wt. % of a fatty alcohol, ester thereof or a mixture thereof.

In another embodiment of the present invention, a topical cream comprising (a) about 0.005 wt. % to about 1.0 wt. % hydrocortisone butyrate; and (b) two or more surfactants which provides a combined HLB to the composition, of about less than 14 preferably between 9 to 11, more preferably 9.5 to 10.8, wherein the concentration of a fatty alcohol, ester or a mixture thereof is less than 6.0 wt. % preferably from about 2.0 wt. % to 4.0 wt. %.

The term “relief” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The topical cream, herein described, may be prepared by the use of conventional methods for the manufacture of ointments and creams. Conventional techniques employing microscopic examination of creams sometimes shows a picture of a complex emulsion, and sometimes an oil-in-water emulsion depending on the intensity of the mixing procedure. In the case of a complex emulsion, the continuous phase is the oily components in some parts of the emulsion and in other parts the continuous phase is water. The topical creams have an occlusive action when applied to skin due to the high oil content and the skin becomes moist and therefore there is better penetration by the therapeutic agent employed therein. Moreover, the topical creams, in contrast with ointments, are non-greasy and are readily removable from the skin or other materials which come into contact with the creams.

The “fatty alcohols and esters thereof” which are incorporated in the topical creams of the invention are those commonly used in the preparation of ointments and creams. Representative examples include stearyl alcohol, cetyl alcohol, cetyl stearyl alcohol, myristyl alcohol, glycerin monostearate, glyceryl stearate, PEG-100 stearate and the like and mixtures thereof Octyldodecanol, a mixture of stearyl alcohol and cetyl alcohol, is preferably used and the acid moiety may be supplied in a filly esterfied compound or one which is less than fully esterfied, e.g., glyceryl monostearate and glyceryl di-stearate, respectively. The fatty esters for use herein can be a fatty acid (e.g., stearic acid) esterified with a lower chain alcohol such as a C₁-C₁₂ alcohol, e.g., methyl-, ethyl-, n-propyl-, isopropyl- and butyl ester of stearic acid and the like. The fatty esters for use herein can also be a fatty acid esterified with a polyol. Suitable polyols include those polyols containing from two to about 10 carbon atoms and from two to six hydroxyl groups. Useful polyols include, but are not limited to, sorbitol, ethylene glycol, diethylene glycol, polyethylene glycol, 1 ,2-propanediol, 1 ,3-propanediol, 1,2-butanediol, 1,3-butanediol, 2,3-butanediol, 2-ethyl-1,3-propanediol, 2-ethyl-2-butyl-1,3-propanediol, neopentyl glycol, 2,2,4-trimethyl-1,3-pentanediol, trimethylolpropane (TMP), pentaerythritol and the like and mixtures thereof.

The fatty materials which are incorporated in the topical creams of the present invention are those commonly used in the preparation of ointments and creams. Preferably, a mixture of fatty compounds which may be solid, semi-solid and/or liquid at ambient temperature is used in the preparation of the topical creams. The fatty compounds may be, for example, waxes such as white soft paraffin, and liquid paraffin, fatty alcohols and esters, vegetable oils such as cottonseed, coconut oil, soybean oil and peanut oil, mineral oils, liquid silicones and the like and mixtures thereof Preferably, a combination of liquid paraffin and white soft paraffin is employed.

The amount of hydrophilic surfactant which may be liquid or solid used in the preparation of the topical creams of the invention is preferably from about 1.0% to about 5.0%, by weight of the final composition. Examples of useful surfactants include CEMATOCROGEL®1000 (i.e., polyethylene glycol (1000) monocetyl ether), POLYSORBATE®60 (i.e., polyoxyethylene (20) sorbitan monostearate), POLYSORBATE®80 or TWEEN®80 (i.e., polyoxyethylene (20) sorbitan monooleate, sorbitan monooleate), PEG-20 glyceryl stearate, PPG-10 glyceryl stearate, Arlacel®165 (glyceryl stearate and PEG 100 stearate), Ceteth®-20, Brij® and the like and mixtures thereof Advantageously, only one surfactant may be employed herein.

In one of the aspects of present invention, the use of two or more surfactants, indicates that two or more surfactants of substantially a same or different HLB values are employed, in suitable ratios, such that it provides the combined HLB values to the composition of less than about 14.

For the purpose of the present invention, the combined HLB for two or more surfactants is determined as follows “A composition comprising two surfactants X and Y, wherein, surfactant X (HLB=15) is 70% and surfactant Y (HLB=4.3) is 30%, of the total weight surfactants X and Y. The combined HLB of the composition would be 11.8 X at 70% multiplied by 15.0=10.5, Y at 30% multiplied by 4.3=1.3, giving the combined HLB , as the sum of10.5+1.3=11.8).

If desired, it may be useful to include in the topical creams of the invention a buffering agent to maintain a desired pH value. Suitable buffering agents include a combination of (i) citric acid and sodium citrate, (ii) phosphoric acid and sodium phosphate, or (iii) lactic acid and sodium lactate, as appropriate for the desired pH. Where the therapeutic agent is hydrocortisone-17α-butyrate, a slightly acidic environment of pH about 3.5 to about 4.5 is needed to prevent hydrolysis of the butyrate and a combination of citric acid and sodium citrate is particularly useful for this purpose.

The topical creams of the invention may also contain a preservative to prevent, for example, bacterial attack. Suitable preservatives include butylparaben, propylparaben, chlorocresol, sorbic acid, benzoic acid and the like and mixtures thereof

As mentioned above, the topical creams of the invention may be prepared by conventional means, but a preferred method involves mixing the ‘fatty’ components, e.g., cetyl/stearyl alcohol, liquid paraffin and white soft paraffin with the hydrophilic non-ionic surfactant such as Ceteth®-20, and heating the mixture at, for example, from about 70° C. to about 80° C. Water (preferably de-ionised), buffering agent and preservative are mixed separately in a suitable ointment-mixer and heated, for example, at about 70 to about 80° C., until a solution is obtained. A small portion of the resulting solution is separated and cooled to ambient temperature, and then the hydrocortisone butyrate is added.

The liquid fatty composition at elevated temperature of about 70° C. to about 80° C. is added to the larger portion of the aqueous solution and then, after vigorous stirring under reduced pressure to avoid air bubbles in the final topical cream. The aqueous solution or suspension containing the therapeutic agent is then added at normal pressure. After vigorously mixing the mass at about ambient temperature for an appropriate length of time and once again under reduced pressure, a fatty topical cream is obtained.

The temperature of the final mixing of the components of the topical cream and the agitation involved can affect the form of the emulsion (oil-in-water and/or water-in-oil) thus obtained. Preferably, it is effected at about 20° C. to about 25° C. with vigorous agitation.

The following examples merely illustrate the lotion compositions of the invention and are not to be construed as limiting the scope of the invention. Unless indicated otherwise, all weight percentages are based on the total weight of the composition

### EXAMPLES

### Example 1

A topical cream in accordance with the present invention was prepared having the following ingredients and amounts as set forth below in TABLE 1.

TABLE 1

| Sr. | | |
|---|---|---|
| No. | Composition | % w/w |
| 1. | Hydrocortisone Butyrate | 0.10 |
| 2. | Mineral oil | 15.0 |
| 3. | Cetostearyl alcohol | 6.00 |
| 4. | Octyldodecanol | 10.0 |
| 5. | White Petrolatum | 35.0 |
| 6. | Ceteth ®-20 | 3.00 |
| 7. | Citric acid anhydrous | 0.18 |
| 8. | Sodium citrate anhydrous | 0.12 |
| 9. | Butylparaben | 0.05 |
| 10. | Propylparaben | 0.10 |
| 11. | Purified Water | 30.45 |

Manufacturing Procedure

1. Oil phase: Cetostearyl alcohol, white petrolatum, and Ceteth®-20 were melted together. Next, mineral oil and octyldodecanol, butylparaben and propylparaben were added and dissolved in the melt. The temperature was maintained at 70° C. to 75° C.

2. Aqueous phase: Citric acid and sodium citrate were dissolved in water. A part of the aqueous phase was separated from the phase and then heated and maintained at a temperature of 70° C. to 75° C.

3. Drug suspension: Hydrocortisone butyrate was dispersed in the separated part of the aqueous phase.

4. Emulsification: The oil phase was added to the aqueous phase slowly and homogenized for 15 minutes. During homogenization the drug suspension was added.

5. Cooling: The product of 4 was maintained under slow stirring at room temperature.

### Example 2

A topical cream in accordance with the present invention was prepared having the following ingredients and amounts as set forth below in TABLE 2.

TABLE 2

| Sr. | | |
|---|---|---|
| No. | Composition | % w/w |
| 1. | Hydrocortisone Butyrate | 0.10 |
| 2. | Mineral oil | 15.0 |
| 3. | Cetostearyl alcohol | 6.00 |
| 4. | Oleyl alcohol | 10.0 |
| 5. | White Petrolatum | 35.0 |
| 6. | Ceteth ®-20 | 3.00 |
| 7. | Citric acid anhydrous | 0.18 |
| 8. | Sodium citrate anhydrous | 0.12 |
| 9. | Butylparaben | 0.05 |
| 10. | Propylparaben | 0.10 |
| 11. | Purified Water | 30.45 |

Manufacturing Procedure

1. Oil phase: Cetostearyl alcohol, white petrolatum, and Ceteth®-20 were melted together. Next, mineral oil and octyldodecanol, butylparaben and propylparaben were added and dissolved in the melt. The temperature was maintained at 70° C. to 75° C.

2. Aqueous phase: Citric acid and sodium citrate were dissolved in water. A part of the aqueous phase was separated from the phase and then heated and maintained at a temperature of 70° C. to 75° C.

3. Drug suspension: Hydrocortisone butyrate was dispersed in the separated part of the aqueous phase.

4. Emulsification: The oil phase was added to the aqueous phase slowly and homogenized for 15 minutes. During homogenization the drug suspension was added.

5. Cooling: The product of 4 was maintained under slow stirring at room temperature.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

### Example 3

A topical cream in accordance with the present invention was prepared having the following ingredients and amounts as set forth below in TABLE 3.

TABLE 3

| Sr. | | |
|---|---|---|
| No. | Composition | % w/w |
| 1. | Hydrocortisone Butyrate | 0.10 |
| 2. | Mineral oil | 18.0 |
| 3. | Cetostearyl alcohol | 3.00 |
| 4. | White Petrolatum | 42.0 |
| 5. | Arlacel ®165 | 5.00 |
| 6. | Sorbitan mono stearate (Span 60) | 0.80 |
| 7. | Citric acid anhydrous | 0.18 |
| 8. | Sodium citrate anhydrous | 0.12 |
| 9. | Butylparaben | 0.05 |
| 10. | Propylparaben | 0.10 |
| 11. | Purified Water | 30.65 |

Manufacturing Procedure

1. Oil phase: Cetostearyl alcohol, white petrolatum, and Arlacel® 165 were melted together. Next, mineral oil and sorbitan monostearate, butylparaben and propylparaben were added and dissolved in the melt. The temperature was maintained at 70° C to 75° C.

2. Aqueous phase: Citric acid and sodium citrate were dissolved in water. A pair of the aqueous phase was separated from the phase and then heated and maintained at a temperature of 70° C. to 75° C.

3. Drug suspension: Hydrocortisone butyrate was dispersed in the separated part of the aqueous phase.

4. Emulsification: The oil phase was added to the aqueous phase slowly and homogenized for 15 minutes. During homogenization the drug suspension was added.

5. Cooling: The product of 4 was maintained under slow stirring at room temperature.

### Example 4

A topical cream in accordance with the present invention was prepared having the following ingredients and amounts as set forth below in TABLE 4.

TABLE 4

| Sr. | | |
|---|---|---|
| No. | Composition | % w/w |
| | Hydrocortisone Butyrate | 0.10 |
| | Mineral oil | 18.0 |
| | Cetostearyl alcohol | 3.00 |
| | White Petrolatum | 42.0 |
| | Arlacel ® 165 | 5.00 |
| | Sorbitan mono oleate (Span ® 80) | 0.80 |
| | Citric acid anhydrous | 0.18 |
| | Sodium citrate anhydrous | 0.12 |
| | Butylparaben | 0.05 |
| | Propylparaben | 0.10 |
| | Purified Water | 30.65 |

Manufacturing Procedure

1. Oil phase: Cetostearyl alcohol, white petrolatum, and Arlacel® 165 were melted together. Next, mineral oil and sorbitan mono oleate, butylparaben and propylparaben were added and dissolved in the melt. The temperature was maintained at 70° C. to 75° C.

2. Aqueous phase: Citric acid and sodium citrate were dissolved in water. A part of the aqueous phase was separated from the phase and then heated and maintained at a temperature of 70° C. to 75° C.

3. Drug suspension: Hydrocortisone butyrate was dispersed in the separated part of the aqueous phase.

4. Emulsification: The oil phase was added to the aqueous phase slowly and homogenized for 15 minutes. During homogenization the drug suspension was added.

5. Cooling: The product of 4 was maintained under slow stirring at room temperature.

1. A topical cream comprising (a) about 0.005 to about 1.0 wt. % hydrocortisone butyrate, (b) about 6.0 wt. % to about 24.0 wt. % a fatty alcohol, fatty alcohol ester or a mixture thereof; (e) about 50 wt. % to about 80 wt. % fatty component, (d) about 1.0 to about 5.0 wt. % of at least one surfactant; and the balance being water.

2. The cream of claim 1, wherein the hydrocortisone butyrate is present at about 0.1 wt. %.

3. The cream of claim 1, wherein the fatty alcohol, ester thereof or a mixture thereof is present in a range from about 10.0 wt. %. to about 20.0 wt. %.

4. The cream of claim 1, wherein the cream is an oil-in-water fatty cream.

5. The cream of claim 1, wherein the fatty alcohol is cetostearyl alcohol, octyldodecanol, oleyl alcohol or mixtures thereof

6. A topical cream comprising (a) about 0.005 wt. %, to about 1.0 wt % hydrocortisone butyrate; (b) about 6.0% cetostearyl alcohol, (c) about 10% wt. %.to about 15% oleyl alcohol, (d) about 50% to about 80 wt. % fatty component, (e) about 1.0 wt. % to about 7.0 wt. % of at least one surfactant; and the balance being water.

7. The cream of claim 6, wherein the hydrocortisone butyrate is present at about 0.1 wt. %.

8. The cream of claim 6, wherein the cream is an oil-in-water fatty cream.

9. A topical cream, comprising (a) about 0.005 wt. %. to about 1.0 wt. % hydrocortisone butyrate; (b) less than about 6.0 wt. % a fatty alcohol, ester thereof or a mixture thereof; (c) about 50 wt. %. to about 80 wt. % fatty component; (d) about 1.0 wt. %. to about 7.0 wt. % of two or more surfactants; and the balance being water, wherein the two or more surfactants provides the HLB of less than about 14.

10. The cream of claim 9, wherein the cream is an oil-in-water fatty cream.

11. The oil-in-water fatty cream of claim 10, wherein the surfactant is selected from sorbitan monooleate, sorbitan monostearate, glyceryl stearate and PEG-100 stearate

12. The oil-in-water fatty cream of claim 10, wherein the surfactants are sorbitan monooleate, glyceryl stearate and PEG-100 stearate.

13. The oil-in-water fatty cream of claim 10, wherein the surfactants are sorbitan mono stearate, glyceryl stearate (and) PEG-I 00 stearate.

14. The oil-in-water fatty cream of claim 10, wherein the combined HLB value is between about 9.5 to about 11.
